(19) United States
(12) Patent Application Publication
(10) Pub. No.: US 2007/0068512 A1
(43) Pub. Date: Mar. 29, 2007

(54) Adjustable truss

(76) Inventor: Pang-Ching Chiang, Taipei (TW)

Correspondence Address: PATENTTM.US, P. O. BOX 82788, PORTLAND, OR, 97282-0788, US

(21) Appl. No.: 11/238,593

(22) Filed: Sep. 28, 2005

(51) Int. Cl. A61F 5/28

(52) U.S. Cl. 128/100.1

(57) ABSTRACT

A truss has a belt, two connectors, two straps and at least one pad. The belt has a front, a back, two ends, an outer side, an inner side, and a fastener. The connectors are attached to the belt, and each connector has a fixed pad connector. The straps are attached respectively to and extend down from the connectors and attach to the back of the belt. The at least one pads is attached to the appropriate connector and has a detachable pad connector. The detachable pad connector on the pad selectively attaches to the appropriate fixed pad connector to hold the pad on the belt. The pad connectors allow the pads to be adjusted, and the truss fits a user and is comfortable.

### BACKGROUND OF THE INVENTION

1. Field of the Invention

The present invention relates to a truss, especially to a truss that can be adjusted to fit people with different statures.

2. Description of Related Art

With reference to FIG. 7, a conventional truss is worn around a person's hips, presses against a hernia to prevent a strangulated hernia and comprises a belt (40), two pads (41), two connectors (42), two straps (43), and a restrictor strip (44). The belt (40) is elastic, is worn around a person's hips (not shown) and has an inner side (not numbered) and an outer side (not numbered), a front (not numbered), a back (not numbered) and two sides (not numbered). The pads (41) are attached to the inner side of the belt (40) and press against the abdomen just above a person's scrotum. The connectors (42) are sewn to the front of the belt (40), correspond respectively to the pads (41) and are strong non-elastic material, and cach connector may comprise two patches (not numbered). The straps (43) are elastic, are connected respectively to the connectors (42), extend down, connect to the back of the belt (40), may be adjustable and hold the belt (40) and pads (41) in position when a person wears the truss. The restrictor strip (44) is a strip of non-elastic material is sewn to the belt (40) and the connectors (42) to hold the connectors (42) in position relative to each other.

The conventional truss has the following shortcomings. The pads (41) are sewn directly to the belt (40) and cannot be adjusted to accommodate differences in users' bodies. The restrictor strip (44) keeps the pads (41) from separating excessively but also keeps the pads (41) from being adjusted relative to each other and makes the truss uncomfortable.

The present invention provides an adjustable truss to obviate or mitigate the aforementioned problems.

### SUMMARY OF THE INVENTION

The main objective of the present invention is to provide an adjustable truss that fits a user's body and feels comfortable.

The adjustable truss in accordance with the present invention comprises a belt, two connectors, two straps, at least one pad and an optional restrictor strip. The belt may be elastic and has a front, a back, two ends, an outer side, an inner side, a fastener and two optional rings. The connectors are attached to the belt, and each connector has a fixed pad connector. The straps are attached respectively to and extend down from the connectors and are attached to the back of the belt, and each strap has two ends and an optional fastener. One end is connected to the connector on the front of the belt. The other end is connected to the back of the belt and extends through the corresponding ring. The fastener is on the end of the strap connected to the back of the belt, can be used to adjust the strap and may be a buckle or a hook and pile connector. The at least one pad is attached to the appropriate connector and has a detachable pad connector and an optional strap loop. The detachable pad connector on the pad selectively attaches to the appropriate fixed pad connector to hold the pads on the belt. The pad connectors allow the pads to be adjusted. The adjustable restrictor strip is attached to the outer side of the belt and the connectors and has two straps and a fastener to allow further adjustment of the belt and the pads.

Other objectives, advantages and novel features of the invention will become more apparent from the following detailed description when taken in conjunction with the accompanying drawings.

### BRIEF DESCRIPTION OF THE DRAWINGS

FIG. 1 is a rear perspective view of a truss in accordance with the present invention;

FIG. 2 is a front perspective view of the truss in FIG. 1;

FIG. 3 is an operational front view of the truss in FIG. 1;

FIG. 4 is an operational rear view of the truss in FIG. 1;

FIG. 5 is an operational front view of the truss in FIG. 1 with the pads adjusted vertically to different positions;

FIG. 6 is another operational front view of the truss in FIG. 1 with the pads adjusted horizontally to different positions; and

FIG. 7 is a perspective view of a conventional truss in accordance with the prior art.

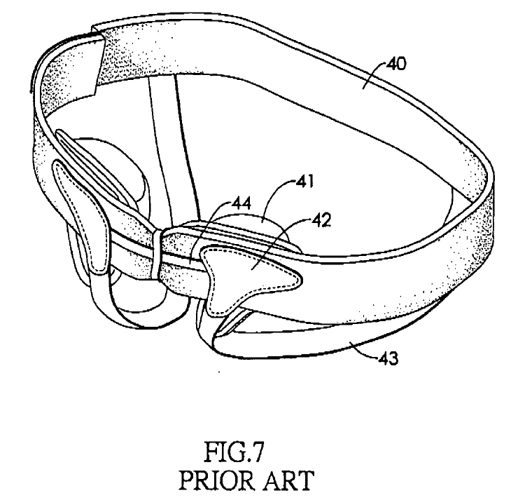

### DETAILED DESCRIPTION OF THE PREFERRED EMBODIMENT

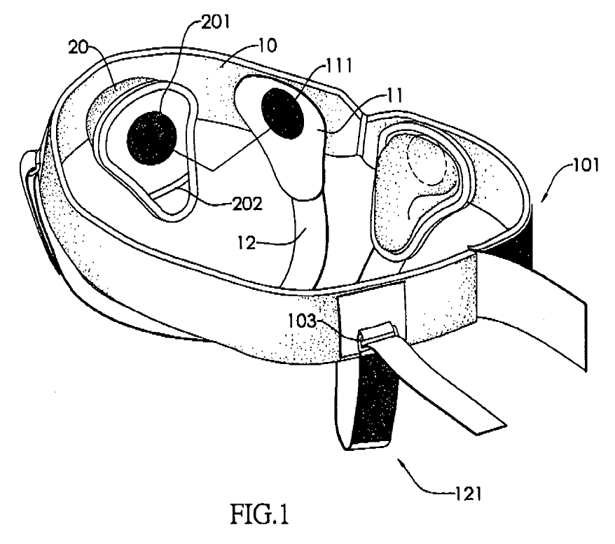

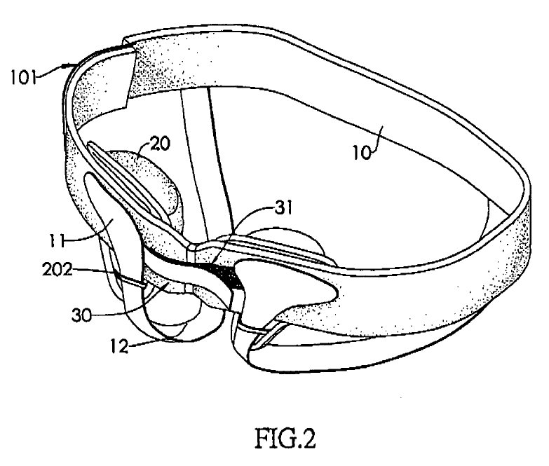

With reference to FIGS. 1 and 2, a truss in accordance with the present invention comprises a belt (10), two connectors (11), two straps (12), at least one pad (20) and an optional adjustable restrictor strip (30).

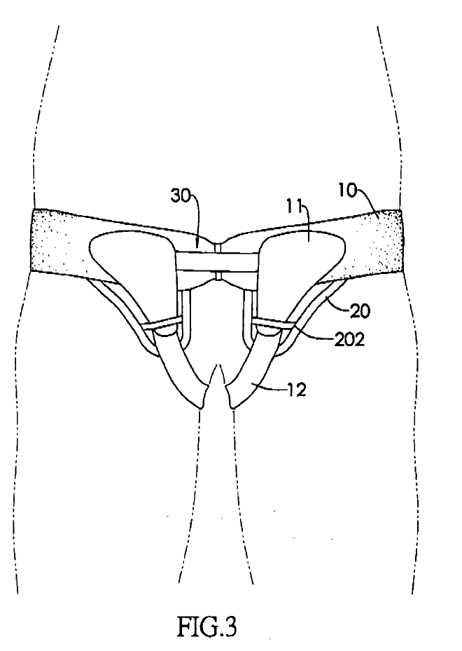

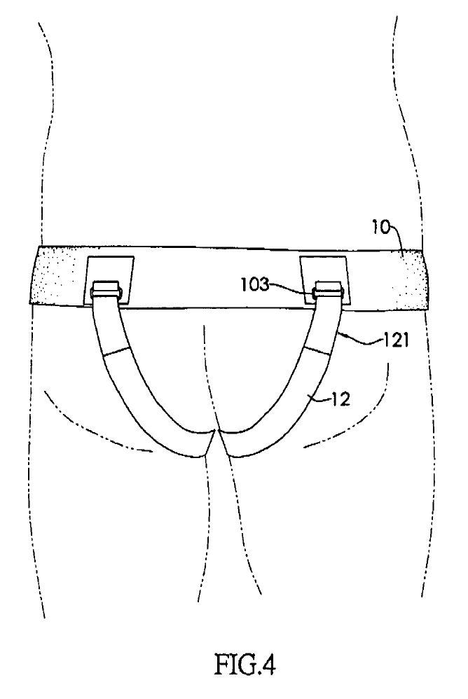

With further reference to FIGS. 3 and 4, the belt (10) may be elastic, is worn around a person's hips (not numbered) and has a front, a back, two ends, an outer side, an inner side, a fastener and two optional rings (103). The fastener is attached to at least one of the two ends, fastens the two ends together and may be implemented with a buckle (not shown), a bayonet connector (not shown) or a hook and pile connector (101). The rings (103) are attached to the back of the belt (10) on the outer side.

The connectors (11) are attached to the front of the belt (10), and each connector (11) has a fixed pad connector (111). The fixed pad connectors (111) are on the inner side of the belt (10).

The straps (12) are attached respectively to and extend down from the connectors (11) and are attached to the back of the belt (10), and each strap (12) has two ends and an optional fastener (121). One end is connected to the connector (11) on the front of the belt (10). The other end is connected to the back of the belt (10) and extends through the corresponding ring (103) to connect the strap (12) to the belt (10) when the rings (103) are attached to the belt (10). The fastener is on the end of the strap (12) connected to the back of the belt (10), can be used to adjust the strap (12) and may be a buckle (not shown) or a hook and pile connector (121).

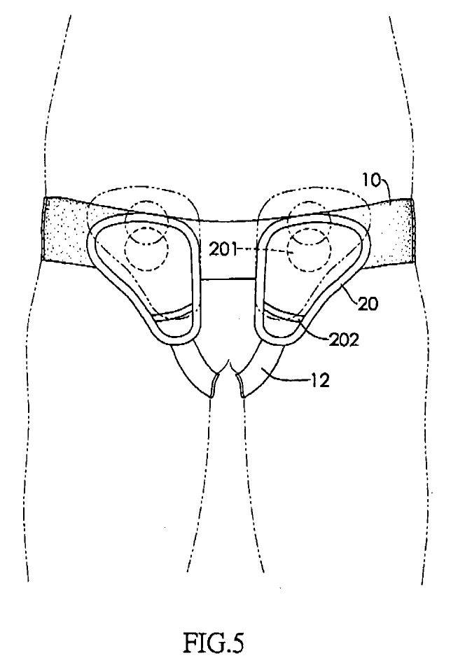

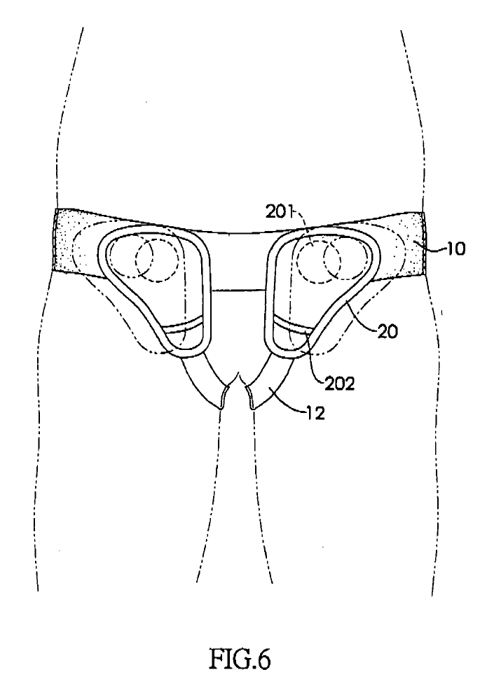

The at least one pad (20) is attached to the corresponding connector (11), and each pad (20) has a detachable pad connector (201) and an optional strap loop (202). With further reference to FIGS. 5 and 6, the detachable pad connectors (201) respectively on the pads (20) selectively attach respectively to the fixed pad connectors (111) to hold the pads (20) on the belt (10) and allow the pads (20) to be adjusted. The strap loops (202) respectively on the pads (20) allow the corresponding straps (12) to extend respectively through strap loops (202) and help hold the pads (20) in position.

The adjustable restrictor strip (30) is attached to the outer side of the belt (10) and the connectors (11) and has two straps and a fastener to allow further adjustment of the belt (10) and the pads (20). The fastener is attached to the straps, connects the straps together, adjusts the straps and may be a buckle (not shown) or a hook and pile connector (31).

The truss as described has the following advantages. The detachable pad connectors (201) allow the pads (20) to be adjusted, and the adjustable restrictor strip (30) adjusts the pads (20) relative to each other to fit the user's body.

Even though numerous characteristics and advantages of the present invention have been set forth in the foregoing description together with details of the structure and function of the invention, the disclosure is illustrative only. Changes may be made in detail especially in matters of shape, size, and arrangement of parts within the principles of the invention to the full extent indicated by the broad general meaning of the terms in which the appended claims are expressed.

What is claimed is:

1. A truss comprising
a belt adapted to be worn around a person's hips and having
a front;
a back;
two ends;
an outer side;
an inner side; and
a fastener attached to at least one of the ends and fastening the two ends together;
two connectors attached to the front of the belt, and each connector having a fixed pad connector mounted on the inner side of the belt;
two straps attached respectively to and extending down from the connectors and attached to the back of the belt, and each strap having two ends with one end connect to one of the connectors on the front of the belt, and the other end connected to the back of the belt; and
at least one pad respectively attached to at least one of the connectors, and each one of the at least one pad having a detachable pad connector attaching to one of the fixed pad connectors.

2. The truss as claimed in claim 1, further comprising an adjustable restrictor strip attached to the outer side of the belt and the connectors and having
two straps; and
a fastener attached to at least one of the straps to connect the straps together and to adjust the straps.

3. The truss as claimed in claim 1, wherein each pad further has a strap loop to allow the corresponding strap to extend through the strap loop.

4. The truss as claimed in claim 1, wherein the belt further has two rings attached to the back of the belt on the outer side, and each strap extends through one of the rings to connect the strap to the belt.

5. The truss as claimed in claim 1, wherein the belt is elastic.

6. The truss as claimed in claim 1, wherein the fastener on the belt is implemented with a hook and pile connector.

7. The truss as claimed in claim 1, wherein each strap further has a fastener on the end of the strap connected to the back of the belt and used to adjust the strap.

8. The truss as claimed in claim 2, wherein the fastener on the adjustable restrictor strip is a hook and pile connector.

9. The truss as claimed in claim 7, wherein the fastener on each strap is a hook and pile connector.
